(19) United States
(12) Patent Application Publication
(10) Pub. No.: US 2023/0304199 A1
(43) Pub. Date: Sep. 28, 2023

(54) TEXTILE INSERT FOR MEDICAL PURPOSES, AND METHOD FOR PRODUCING SAME

(71) Applicants: MDB TEXINOV, Saint Didier De la Tour (FR); DELTAVAL, Lyon (FR)

(72) Inventors: Eric Mignot, Velanne (FR); Sébastien Leo, Bilieu (FR); Maud Peyrachon, Voiron (FR); Nadège Boucard, Pont De Beauvoisin (FR); Jacques Tankere, Meximieux (FR)

(21) Appl. No.: 18/204,129

(22) Filed: May 31, 2023

Related U.S. Application Data
(62) Division of application No. 16/649,087, filed on Mar. 19, 2020.

(30) Foreign Application Priority Data
Oct. 4, 2017 (FR) 1759264

(51) Int. Cl. D04B 21/20; A61F 2/00

(52) U.S. Cl. CPC D04B 21/205; A61F 2/0077; A61F 2/00; A61F 2/0063; A61F 2/2445

(57) ABSTRACT

This three-dimensional knitted textile insert is obtained in a single step by warp knitting technology, on a double-needle Raschel machine or on a crocheting machine. It includes in the production direction at least two tubes extending parallel to one another, and separated from one another by a binding area at the level of which the sheets resulting from the knitting are interlinked.

### FIELD OF TECHNOLOGY

The invention pertains to the field of textile inserts, particularly for a medical use. It concerns the actual implants as well as textile structures intended to cover implants of more conventional nature.

The fitting of a prosthesis or of an implant goes along with an inflammatory response, the latter being all the more acute as the individual's biological tolerance is low. The forming of a good scar tissue then requires an intense fibroblastic activity.

The fibroblastic activity phase is preceded by a critical period during which the stability of the implant should be ensured by anchor points. The implant should thus allow a good adhesion to tissues, to favor the development of a strong cell colonization which accompanies the forming of a high-quality scar tissue.

### BACKGROUND

The implants currently used are generally manufactured from either metallic materials such as stainless steel, titanium and its alloys, or from plastic materials. If such implants then have excellent mechanical properties, the low adhesion of tissues is a major limit to their implementation. Indeed, due to this low adhesion, the cell colonization of the implant and thus, as a corollary, the forming of a high-quality scar tissue are deficient. Further, in the case of metal implants, the risk of toxicity due to the accumulation of metal ions due to corrosion is not absent.

To overcome this problem, various techniques have been developed, aiming at favoring the integration of implants of metal or plastic type. Among these, some comprise grafting or coating with chemical products the surface of the concerned implant. Such products induce the forming of microporous structures at the surface of the implant, such as for example described in document WO 2014/024171. There are many examples of treatment of implants by seeding, vapor deposition, electrolysis, and deposition of various chemical products. Certain techniques further advocate the creation of microporosities at the surface of the implant by chemical etching, as described in document EP 0.388.576.

Such techniques of surface treatment of the implant however require a systematic study of the compatibility between the deposited layer and the material of the implant, be it metallic or made of plastic material. A unicity of the deposited material/implant pair can indeed often be observed, on which the risks of release of the deposited material depend, which release is more and more restricted by the standards in force, the latter even tending towards a zero release risk.

Further, such techniques may turn out being expensive and complex to implement, and the selected coating method does not necessarily adapt to the sometimes complex shapes of the concerned implant.

It has already been provided to replace the metal implants with ceramic implants. With such ceramic implants, the creation of micro and nanoporosities by oxidation of the surface, resulting in a good cell adhesion, such as for example described in document US 2010/274361, could indeed be observed.

Such methods are however long and expensive, and the performance of ceramic implants, even though constantly evolving, has not reached the level of that of metal or plastic implants yet.

Another approach comprises using textile implants, either as such, or in association with the previously-discussed materials to cover them or ensure a linking with the biological tissues, possibly easing the securing by the surgeon. These have good colonization capacities, the latter being favored on the one hand by the materials used for their manufacturing (PET, polypropylene, . . . ), on the other hand by their structure made of yarns, and finally by the calibration of their stitch, defining pores, the size of said pores being determining for the rigidity as well as for the anchoring of scar tissues on the implant.

The textile portions of the implants are formed either of a woven structure, or in a knit structure. They often require a phase of hand cutting and assembly, which is expensive, and which raises a reproducibility issue since it depends on the operator.

The object targeted by the present invention is to provide an insert implantable in the form of a microtubular textile. The invention thus comprises the forming of inserts, in the form of multi-microtubes, allowing a low inflammation accompanied by a strong fibroblastic activity.

The insert is obtained in a single step, has an increased capacity of being colonized, while allowing the insert to be handled more easily by the surgeon and thus making it simpler to use. The invention thus enables to suppress or to decrease the hand manufacturing phase, which results in better reproducibility and decreases costs.

### SUMMARY OF THE DISCLOSURE

Thus, the invention aims at the forming of a three-dimensional knitted textile insert. This insert appears in the form of a multi-microtubes capable of being used either to cover tubular implants, or of complex shape and as a corollary to favor the colonization around said implant, or to favor the securing of the implant to the biological tissue by the surgeon, or to be directly used as an implant.

The three-dimensional knitted textile insert is obtained in a single step by the warp knitting technology, on a double-needle Raschel machine or on a crocheting machine. The obtained insert comprises, in the production direction, at least two parallel tubes, separated by an area at the level of which the sheets resulting from the knitting on the two needle rows are interlinked. These at least two tubes define a main tube and at least one side tube.

A straight yarn or an assembly of capstan-mounted yarns, which thus does not loop, a band, optical fibers, or any other material having length as a main dimension (that is, the main dimension of the insert), a rod, a cord, or a rigidifying element, intended to ease the implementation of the insert implantation, is introduced into at least one of the side tubes, directly on the knitting machine, that is, on forming of the insert.

The use of a warp knitting double-needle Raschel machine or of a crocheting machine enables, on the one hand, to simultaneous manufacture parallel or substantially parallel tubes, and on the other hand, to create bindings or weaves which determine a stitch structure comprising a certain opening or porosity, which is an important parameter to favor the subsequent colonization of the implant.

Advantageously, the porosity may be varied from one tube to the other by varying the size and the structure of the yarns used, which may be mono- or multi-filaments, but also the feeding of said yarns in the machine.

The opening of the pores may be in the range from 0.05 to 3 millimeters in its largest dimension to ease the colonization and the hold of the implant when it is placed by the surgeon. The pore dimension results from the selection of the weave, the thickness of the yarn, and the stitch density.

The at least two tubes, respectively main and side tubes, thus obtained, may have identical or different diameters.

According to the invention, one of the microtubes, be it positioned or not at on the border of the insert, is open. In other words, no binding yarn loops to define the tube in question. The sheets resulting from the two needle rows of the machine are free and are capable of forming areas of sewing of the insert around another element, such as for example, a metal tube made of nickel, titanium, or other, a catheter or an ancillary.

According to the invention, one of the at least two tubes, in the case in point the main tube, has a diameter much greater than the diameter of the other tube(s). The tube of greater diameter is intended to receive a catheter, a tube of Nitinol type (alloy of nickel and of titanium), enabling to secure a catheter or another functional element during the intervention, or an artificial element for replacing an organ, such as for example, a heart valve prosthesis ring.

Typically, the diameter of the considered tube is at least 3 millimeters. It may reach 15 millimeters, or even be larger according to the envisaged applications. The diameters of the side tubes is, as already mentioned, smaller and typically in the range from 0.5 to 2 millimeters. The diameter of the tubes is determined by the selection of the machine gauge, the threading on each bar of said machine and by the number of needles at work.

According to the invention, the tubes forming the insert are either tangent to each other, or separated from each other or from one another by a flat area resulting from the binding of the sheets originating from each needle row or from the absence of yarn in one of the two needle rows.

According to an embodiment of the invention, the insert comprises three tubes, respectively a main central tube having a nominal diameter of at least 3 millimeters, of the previously-discussed type, while the other tubes have smaller diameters, and typically in the range from 0.5 to 2 millimeters. In the case where the multi-microtubes is formed of three tubes, there then is a main central tube to which are added two smaller tubes called side tubes, positioned in diametrically opposite fashion on either side of the central tube.

The technique implemented to form the insert of the invention enables to directly introduce, on manufacturing of the insert, in the production direction, rods or cords, and generally rigidifying elements, which end up in the side tubes of smaller diameter. Thereby, the introduction of the insert thus formed at the level of its implantation location is eased. As a corollary, the forming of a suture area is favored.

As mentioned hereabove, the invention enables to introduce in a single step, into the tubes of the insert (preferably the side tubes, that is, those located on the edges), a straight yarn or an assembly of non-looping yarns (capstan), a band, for example made of polymer or Nitinol, one or a plurality of optical fibers, or any other material having length as a main dimension.

The secondary or side tubes are preferably formed with the same weave as that of the central tube, the geometry of the textile insert being accordingly constant and thus allowing a homogeneous colonization.

According to the complexity of this weave, from 6 to 16 bars of the warp knitting double-needle machine are used to form the invention, thus enabling to vary the number of tubes and their respective diameter.

According to an embodiment of the invention, the binding area between the different tubes is narrow (from 1 to 2 stitch columns). This binding area is formed in the same weave as the other portions of the tubes to ensure a constant porosity of the textile, and thus a homogeneous colonization.

As a variation, the forming of an area of connection between tubes is flat and has a width in the range from 5 millimeters to several centimeters. Thereby, it is possible, with the insert of the invention, to adapt to the practitioner's needs.

According to another embodiment, the sheets resulting from the knitting are interlinked at least on one of their edges in the production direction. There thus is the forming of fins which are used as a support for the suture or the assembly of the insert with other elements of the final medical device. The width of the fins thus formed may vary according to the binding. As a corollary, the assembly of a plurality of inserts of the invention to one another may also be performed via the fins thus defined, particularly by sewing.

The present invention may also see its structure locally reinforced, either by weave effects or the play of the bars of the machine, or by locally modifying the stitch density by electronic driving of the bars and stitch densities, which can thus vary. The reinforcement area thus created may remain identifiable due to the use of color yarns. Such a reinforcement may thus be local, particularly at the level of the area(s) submitted to an effort when the insert is implanted. It may also be used to optimize the seaming or the suture of the insert once implanted.

According to the functions assigned to the implantable insert, one or a plurality of the tubes may be solid, that is, the volume defined by the considered tube(s) is filled with so-called “pile” yarns. These yarns are the usual binding yarns distributed over one or a plurality of bars of a double-needle machine. These yarns alternately pass, due to the machine movement, from one needle row to the other to create a textile exhibiting a thickness.

The different mechanical properties are obtained by the selection of the weave.

Further the elasticity of the lengthening of the insert in the cross-machine direction or in the production direction is possible and may be settable by the selection of the weave, and this, for variable degrees by the selection of the well-known weaves, such as for example, chain, halftone chain, satin.

Further, the selection of the weave makes the textile forming the insert ladderproof.

According to the invention, the knitted textile insert is made of a polymer of natural original, resorptive or not, or of a synthetic polymer or copolymer, resorptive or not, and the materials may be the following: polypropylene, polyethylene, polyester, polyamides, bio-polyesters (PLA: polylactic acid, PGA: polyglycolic acid, PCL: polycaprolactone, PLGA: polylactique-co-glycolic acid), PDO: polydioxanone, Vectran® (aromatic polyester), aramides, mineral fibers, covered yarns . . . .

The yarns used in such a knitted textile insert may be monofilament with a diameter in the range from 30 to 500 micrometers, or multifilaments having a linear density varying from 10 to 5,000 Dtex.

Further, other yarns are likely to be introduced into the insert of the invention on forming thereof, to give it specific properties. Typically, the yarns may be metallic (for example, made of copper, of nickel, of silver, of gold, of aluminum) or shape memory yarns of Nitinol type or made of polymer (for example, PET (polyetherterephthalate), polypropylene, polyethylene, polytetrafluoroethylene, e-PTFE, PLA, PGA, PCL, PDO, aramides).

Many advantages result from such an insert with microtubes. First, it should be underlined that such an insert is almost complete at the output of the knitting machine, thus limiting hand manufacturing phase(s). Thereby, on the one hand, its production cost is limited and, on the other hand, its reproducibility is made more reliable.

Further, the handling of such an insert by the practitioner is eased, said insert being made easier to handle due to an increased gripping capacity. Performing the intervention is also easier with decreased risks, the securing with sutures or seams of the device covered with textile being simpler for the practitioner.

Further, its porosity guarantees it an increased capacity of being colonized, thereby easing the integration of the insert into the human body.

Finally, its elaborate shape limits subsequent operations of manufacturing around the implant, thus adding a security and cost decrease factor.

### BRIEF DESCRIPTION OF THE DRAWINGS

The way in which the invention may be implemented and the resulting advantages will better appear from the following non-limiting embodiments, in relation with the accompanying drawings.

FIG. 1 is a simplified representation of an insert according to the invention, formed of a triple microtube.

FIG. 2 is a simplified representation of the insert of FIG. 1, where the outer tubes each receive a rigidifying organ, such as a cord.

FIGS. 3 and 4 are simplified views of another embodiment of the invention, this time having one or a plurality of fins on either side of the central tube.

FIGS. 5 and 6 are simplified representations of variants of the insert of the invention, comprising a plurality of parallel tubes arranged either tangentially or quasi-tangentially with respect to one another, or spaced apart by flat portions.

FIG. 7 is a simplified representation of a weave implemented for the forming of a multi-microtubes such as shown in FIGS. 1 and 2.

FIG. 8 is a simplified representation of a weave implemented for the forming of a multi-microtubes such as shown in FIG. 3.

FIG. 9 is a simplified representation of a variation of FIG. 2, having an open central tube.

### DETAILED DESCRIPTION

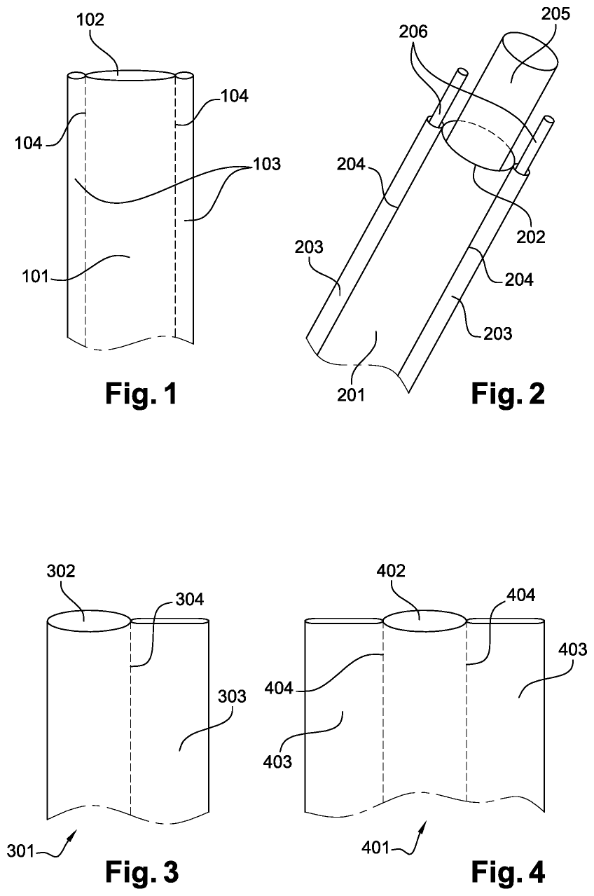

FIGS. 1 and 2 are, as already indicated, simplified views of a triple microtube. The multi-microtube (101) is formed on a warp knitting double-needle machine, typically a Raschel machine, and has a three-dimensional structure.

The following are thus simultaneously formed on said machine:
a main tube (102, 202), in the case in point capable of having a diameter in the range from 3 to 4 millimeters; two side micro-tubes (103, 203), arranged parallel to the main tube (102, 202), and diametrically opposite to each other with respect to said main tube (102, 202); the two tubes (103, 203) are likely to have a typical diameter in the range from 0.3 to 5 millimeters. The respective diameter of the tubes (102, 202; 103, 203) depends on the binding area (104, 204), that is, on the distance between two consecutive binding areas.

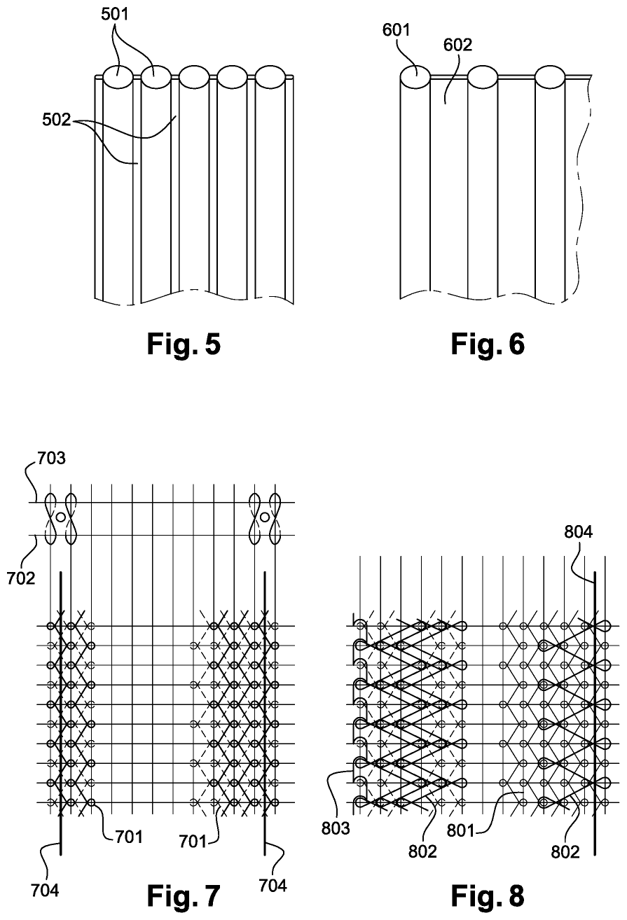

As a corollary, the different tubes extending in the production direction may be either tangent to one another, as illustrated in FIGS. 1 and 2, or separated from one another by a flat area (502, 602) by a variable distance—depending on the requirements imposed for the implementation of the considered insert, and as illustrated in FIGS. 5 and 6.

The main tube (102, 202) may house a catheter or a Nitinol-type tube or an artificial element for replacing a given organ of heart valve ring type, as illustrated in FIG. 2 with the element (205). This catheter or the like is inserted into the tube (102, 202) after the forming of the insert on the knitting machine. Such an insertion is made possible due to the rigidity of the element (205).

This same FIG. 2 also illustrates the introduction of cords or rods or of specific capstan-mounted monofilaments (206) directly introduced on manufacturing on the machine when the rigidity is limited. The rods, cords, or yarns (206) enable to ease the introduction of the insert into the patient's body. They further define an easier and more secure suture area for the securing by the surgeon.

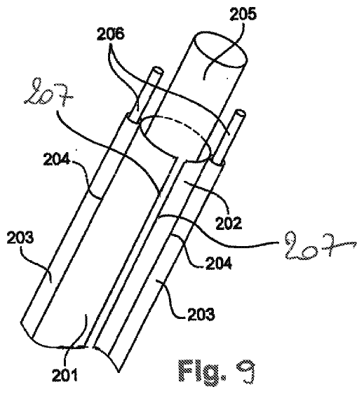

FIG. 9 illustrates a variant of FIG. 2, where the central or main tube (202) is open. The edges defined by the open tube have been materialized with reference (207). To obtain such an open tube, yarns are suppressed at certain locations of one of the two needle rows of the machine. As a variation, it is also possible to obtain such an open tube by adaptation of the threading or of the weave, like for the creation of two widths on a single-needle machine, which techniques is within the abilities of those skilled in the art.

The advantage of such a configuration essentially lies in the possibility of introducing into the insert of the invention an element which cannot be introduced into the machine or which cannot be inserted into the concerned tube after the forming of the textile structure. Such an element is thus associated with the insert of the invention by sewing, and particularly seaming of the two free edges (207) of the concerned tube around said element. Such an element may in particular be formed of a ring of a heart valve, and generally of any element necessary to the functionalizing of the insert.

FIGS. 3 and 4 illustrate a second embodiment. According to an embodiment, fins (303) and (403) are formed on manufacturing of the insert on the machine. Such fins actually result from the binding on the machine of the side tubes to the main tube (302, 402). Such flat portions or fins (303, 403) thus enable to ease, on the one hand, the forming of certain links within the organism, thus favoring thus colonization, and, on the other hand, the practitioner's intervention, the securing of the implant being easier.

FIGS. 5 and 6 show, as already mentioned, alternative embodiments of the tubes illustrated in the previous drawings. The tubes (501) or (601), parallel to one another and oriented in the production direction, are no longer arranged tangentially to one another, but are coupled together by flat portions (502) and (602), resulting from the binding on the machine. The width of the flat areas (502, 602) depends on the bulk of the binding.

FIG. 7 shows a knit obtained by a warp knit technology according to the present invention. This drawing schematically shows a face structure (701) corresponding to the first model of multi-microtubes shown in FIGS. 1 and 2. The work of the yarns on each needle row (702) and (703) can also be observed, which work aims at creating a main tube, with, at its border, the work of the yarns passing from one needle row to the other to enclose the rods and added elements (704), such as for example metal yarns (for example, made of copper, of nickel, of silver, of gold, of aluminum) or shape memory yarns of Nitinol type or of polymer (for example, PET (polyetherterephthalate), polypropylene, polyethylene, polytetrafluoroethylene, e-PTFE, PLA, PGA, PCL, PDO, aramides).

The yarns forming the knit may be mono- or multifilament and are selected from a wide range of natural or synthetic polymer materials, resorptive or not.

This technology further has the advantage of varying the work of the yarns, but also their density in the production direction and in the cross-machine direction, thus determining the porosity of the multi-microtubes, thus easing the grafting of the implant into the organism.

FIG. 8 also shows a knit fabric obtained by warp knitting according to the invention. It relates to the structure corresponding to FIGS. 3 and 4, that is, to the model of a microtube with an adjacent flat portion. The same binding technique is used to form the tube: it is formed on a double needle row while another binding is used to obtain the flat portion. Simple knit (801) and satin (802) bindings are then combined. The latter have partial wefts or weft floats favoring the anchoring in the organism.

The finishing of the insert, particularly at the level of its lateral edges, should be as thin as possible, and advantageously devoid of free yarn ends, so that there is no risk of danger for the organism on introduction thereof. Such a finishing is advantageously formed by the adding of chains (803) at the selvedge of the product, to increase the accuracy of the textile binding construction.

Said selvedges may further receive, by an appropriate looping, inserted yarns (804), such as for example made of metal, of a metallic alloy (Nitinol), or of a polymer, like the yarns (704) of FIG. 7.

What is claimed is:

1. A method for realizing a three-dimensional knitted textile insert, said insert comprising in the production direction at least two tubes extending parallel to one another and separated from one another by a binding area, respectively a main tube of greater diameter and at least one side tube, said method consisting of knitting in a single step by warp knitting technology, on a double-needle Raschel machine or on a crocheting machine, parallel or substantially parallel tubes and creating said binding area, wherein a straight yarn or an assembly of capstan-mounted yarns, which thus does not loop, a band, optical fibers, or any other material having length as a main dimension, a rod, a cord, or a rigidifying element, intended to ease the implementation of the insert implantation, is introduced into one or the side tubes, that is, other than the so-called main tube, in a single step on forming of the insert on the knitting machine.

2. The method for realizing a three-dimensional knitted textile insert according to claim 1, wherein the tubes are tangent to one another or separated from each other or from one another by a planar area.

3. The method for realizing a three-dimensional knitted textile insert according to claim 1, wherein the tube having the largest diameter, called main tube receives a catheter, a Nitinol tube, or an implantable element.

4. The method for realizing a three-dimensional knitted textile insert according to claim 1, wherein the porosity of the insert is defined by the bindings or weaves implemented on the machine, and said porosity differs from one tube to the other of the insert.

5. The method for realizing a three-dimensional knitted textile insert according to claim 5, wherein the opening of the insert pores, defined by the bindings or weaves, is in the range from 0.05 to 3 millimeters in their largest dimension.

6. The method for realizing a three-dimensional knitted textile insert according to claim 1, wherein the yarns are monofilament or multifilament yarns.

7. The method for realizing a three-dimensional knitted textile insert according to claim 1, wherein one of the tubes is open during the knitting.

8. The method for realizing a three-dimensional knitted textile insert according to claim 1, wherein the yarns forming the insert are made of synthetic polymer or copolymer, resorptive or not.

9. The method for realizing a three-dimensional knitted textile insert according to claim 1, wherein the yarns forming the insert are made of polymer of natural origin, resorptive or not.

10. The method for realizing a The three-dimensional knitted textile insert according to claim 1, wherein other yarns are introduced into the insert on forming thereof, to give the insert specific properties, the inserted yarns being metallic or shape-memory yarns of Nitinol type, or made of polymer.
